(19) United States
(12) Patent Application Publication
(10) Pub. No.: US 2019/0314194 A1
(43) Pub. Date: Oct. 17, 2019

(54) A SURGICAL LASER CAPSULORHEXIS SYSTEM AND PATIENT INTERFACE LENS ACCESSORY

(71) Applicants: Alex ARTSYUKHOVICH, Irvine, CA (US); Jin YU, Irvine, CA (US)

(72) Inventors: Alex ARTSYUKHOVICH, Irvine, CA (US); Jin YU, Irvine, CA (US)

(21) Appl. No.: 16/319,298

(22) PCT Filed: Aug. 14, 2017
(86) PCT No.: PCT/US17/46682

§ 371 Date: Jan. 18, 2019

Related U.S. Application Data
(60) Provisional application No. 62/494,544, filed on Aug. 12, 2016.

(51) Int. Cl. A61F 9/008; A61F 9/009; A61B 90/20

(52) U.S. Cl. CPC A61F 9/008; A61F 9/009; A61B 2017/00973; A61F 2009/0087; A61F 2009/00889; A61B 90/20

(57) ABSTRACT

A surgical laser capsulorhexis system includes a laser source, configured to generate a laser cutting beam; a beam guidance system, configured to guide the laser cutting beam from the laser source; a beam focuser, configured to guide the laser cutting beam from the laser source and to generate a focused laser cutting beam, the focused laser cutting beam having a two-dimensional beam pattern; a beam coupler, configured to redirect the focused laser cutting beam; and a patient interface lens, configured to guide the redirected focused laser cutting beam to a tissue surface of a procedure eye.

This application claims priority to U.S. Provisional Patent Application No. 62/494,544, filed on Aug. 12, 2016, which is incorporated by reference for all purposes as if fully set forth herein.

### FIELD OF THE INVENTION

The present invention generally relates to a surgical laser capsulorhexis system and patient interface lens accessory, and a method of performing laser tissue cutting, i.e., a single-shot laser capsulorhexis (capsulotomy).

### BACKGROUND OF THE INVENTION

Cataract surgery is number 1 in volume surgery in the World. Currently there are 3 million cataract surgeries performed each year in US and also 3 million cataract surgeries in Western Europe. The WHO estimates that, by 2020, 32 million cataract surgeries will be performed, up from 12 million in 2000. This is not just due to increased life span and baby boomer demographic population increase. More people use digital devices (tablets, smart phones etc.) and continue active lifestyle in the retirement. As such, more people elect to perform cataract surgery and have functional vision.

Cataract surgery is a process that extracts cataractous (opaque) lens from patient's eye and replaces it with an artificial intra-ocular lens (IOL). A critical step before removing natural human cataractous lens and implanting IOL is capsulorhexis—manual tearing off using special forceps a round opening (about 5 mm in diameter) in anterior surface of the thin transparent film wrapping human lens—the capsule.

The capsular film thickness varies from 2 to 28 micron and is weakly adhering to anterior lens surface. This is a step of cataract surgery that arguably needs most skill and practice from the surgeon. The surgeon needs to learn applying radial tension to a tear in anterior capsule to create a circular tear resulting in a 5 mm round opening. Countless cataract surgeon students are peeling off grape skins in round capsulorhexis fashion, before graduating to pig eyes and finally patients. Even with experienced surgeons reproducibility of capsulorhexis roundness and diameter is still a problem. Some studies suggest that the accuracy in diameter and roundness of capsulorhexis contributes to the long-term stability of IOL in the eye and likelihood of posterior capsule opacification development, which is undesirable.

The current automation solutions for better capsulorhexis are femtosecond lasers, specifically designed for cataract surgery, which are capable of fully automatic capsulorhexis procedure after surgeon immobilizes patient eye in a special suction cup accessory, aligned with laser beam. During the cutting, the system scans the femtosecond laser beam repeatedly to generate a circular cut.

Such laser capsulorhexis results in very round precise diameter capsulorhexis of desirable diameter. The main issue is the cost of femtosecond laser—half a million dollars for a laser. It also has expensive service and maintenance.

Other tools used to help with capsulorhexis include heads-up-displays, integrated with cataract surgeon's view of surgical field that project a superimposed ring image of 5 mm or other diameter to guide surgeon's manual capsulorhexis to an accurate roundness and diameter capsulorhexis. Such idea is implemented in Zeiss Meditec Callisto Eye System as IDIS (Integrated Data Injection System).

Other method also includes inserting an instrument inside the eye with a metal loop that opens to 5 mm diameter circle. A pulse of current heats up the loop and it cauterizes the 5 mm diameter capsulorhexis in anterior capsule. Such device was recently described in US 20150359671 A1.

There is also a device in development that consists of nanosecond thermal laser and a mirror scanner, attached below surgical microscope. The laser beam is scanned to produce a 5 mm diameter circle on anterior capsule. A special contact lens is used to remove effect of corneal refractive power. To localize laser power release the anterior capsule is stained with special dye, absorbing strongly at laser's beam wavelengths. Surgeon uses a contact-type patient interface to push into patient's eye to stabilize the eye and aligns it with the laser scanner, then the surgeon commands laser power release and a ring is burned in anterior capsule. Entire scan takes about a second.

Locating laser under microscope gets in the way of other devices. Microscope attached aberrometer or optical coherence tomograph becomes popular in cataract surgery. Additionally it is desirable to perform a circular capsulorhexis laser cut in one laser shot, so that patient eye is not moved during the cutting.

Accordingly, there is a need for improved devices, systems, and methods that facilitate real-time, intra-surgery surgical laser capsulorhexis by addressing one or more of the needs discussed above.

### SUMMARY OF THE INVENTION

The current invention provides a surgical laser capsulorhexis system that includes a laser source, configured to generate a laser cutting beam; a beam guidance system, configured to guide the laser cutting beam from the laser source; a beam focuser, configured to guide the laser cutting beam from the laser source and to generate a focused laser cutting beam, the focused laser cutting beam having a two-dimensional beam pattern; a beam coupler, configured to redirect the focused laser cutting beam; and a patient interface lens, configured to guide the redirected focused laser cutting beam to a tissue surface of a procedure eye.

In one embodiment, the beam focuser includes a beam-shaping element selected from the group consisting an axicon optical element, a diffractive element, a holographic element, a spatial light modulator, a refractive element, bundle fibers, a ring illuminator, a micro-mirror device, a MEMS based device, and a deformable platform.

In one embodiment, the beam focuser is a micro-mirror device, a MEMS based device, a deformable platform, a galvanometer-based scanner, a polygon scanner, or a resonant PZT scanner.

In one embodiment, the two-dimensional beam pattern is selected from the group consisting of a line, a circle, a ring, an ellipse, a curve, a star, a spiral, a raster, a cross, concentric circles, a constant-radius asterisk, a multiple-radius asterisk, an arcuate shape of various length and angular locations, and a multiply folded path.

In one embodiment, the laser source has an operating wavelength in 0.2-2.2 micron, 0.7-2.5 micron, or 1.8-25 micron wavelength range.

In one embodiment, the surgical laser capsulorhexis system further includes a visible alignment target, configured to facilitate alignment of the redirected focused laser cutting beam into a target region of the tissue surface.

In one embodiment, the visible alignment target is generated by an aiming beam source indicating the location of the redirected focused laser cutting beam on a lens capsule surface.

In one embodiment, the visible alignment target covers a range of 3 mm-15 mm in diameter on the tissue surface.

In one embodiment, the visible alignment target is a ring, a line, a cross, a circle, a donut shape, a dot, an arc, a curve, or a star.

In one embodiment, the surgical laser capsulorhexis system further includes a surgical microscope, and the beam coupler is configured to redirect the focused cutting beam into an optical pathway of the surgical microscope.

In one embodiment, the visible alignment target is located between the procedure eye and the surgical microscope, on one surface of the beam coupler, or the patient interface lens.

In one embodiment, the beam coupler includes a dichroic mirror, a notch filter, a hot mirror, a beamsplitter, a beam coupler, a beam combiner or a cold mirror in a tilted position.

In one embodiment, the beam coupler is a hand-held device.

In one embodiment, the beam coupler and the patient interface lens are integrated.

In one embodiment, the beam focuser and the beam coupler are integrated into one optical block.

In one embodiment, the beam coupler is coupled to the surgical microscope with a defined optical/opto-mechanical relationship.

In one embodiment, the beam coupler is coupled to the surgical microscope by a suspension system, a mechanical frame, a protruding arm, a conical structure, a magnetic member, an elastic member, or a plastic member.

In one embodiment, the patient interface lens is a non-contact lens, positioned by a mechanical coupling to the beam coupler, a mechanical coupling to the surgical microscope, a suspension system, or a lens holder, or is a contact lens configured to be contacted to the procedure eye.

In one embodiment, the patient interface lens is embedded in a stabilizing mechanism, the stabilizing mechanism configured to stabilize the patient interface lens relative to the procedure eye.

In one embodiment, the stabilizing mechanism includes a trocar, a counter weight, an air suction, a friction-based system, or an elastic system.

In one embodiment, the surgical laser capsulorhexis system further includes an footswitch, configured to start laser cutting.

In one embodiment, the aiming beam source has an operating wavelength in 0.4-0.8 micron wavelength range.

In one embodiment, the beam guidance system includes a fiber optical guide and a free space guidance system.

The present invention also provides a method of performing laser tissue cutting that includes: generating a laser cutting beam with a two-dimensional beam pattern; providing a visible alignment target; aligning the laser cutting beam with a target region of a tissue layer of a procedure eye using the visible alignment target; and delivering the laser cutting beam to the target region to complete the laser tissue cutting.

In one embodiment, the method further includes receiving a corresponding control command to deliver the laser cutting beam.

In one embodiment, the method further includes receiving a corresponding control command from a footswitch.

In one embodiment, the method further includes providing a beam coupler; and redirecting the laser cutting beam into an optical pathway of a surgical microscope using the beam coupler.

In one embodiment, the method further includes generating the visible alignment target by using an aiming beam source in 0.4-0.8 micron wavelength range, or generating a visible pattern or mark between the procedure eye and the surgical microscope.

The present invention also provides a method to perform laser tissue cutting that includes: generating a laser cutting beam with a two-dimensional beam pattern; and delivering the laser cutting beam to the target region to complete the laser tissue cutting with a single shot.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 depicts schematically a capsulorhexis laser and contact lens delivery device for precise single laser shot capsulorhexis with laser pulse delivered over optical fiber. In FIG. 1, numeral 1 represents patient; 2—patient's eye; 3 contact lens delivery device; 4—optical fiber; 5—capsulorhexis laser; 6—surgeon; 7—surgical microscope.

FIG. 2 is a diagram illustrating one implementation of a surgical laser capsulorhexis system.

FIG. 3 is a diagram illustrating another implementation of surgical laser capsulorhexis system.

FIG. 4 is a diagram illustrating multiple embodiments of a beam focuser for the surgical laser capsulorhexis system.

FIG. 5 is a diagram illustrating visible alignment targets for surgical laser capsulorhexis system.

FIG. 6 depicts a cross-section of specialized contact lens accessory for a delivery of ring focused laser beam to anterior capsule. In FIG. 6, numeral 1 represents patient's eye; 2—human lens; 3—contact lens (sterile); 4—contact lens handle sleeve (sterile) with optical tube insert (non-sterile); 5—fiber connector receptacle; 6—fiber connector; 7—optical fiber; 8, 9—aspheric lenses; 10—axicon optical element; 11—dichroic mirror with optical band reflecting laser wavelength; 12—patient's cornea wrapped with contact lens concave surface.

In the drawings, elements having the same designation have the same or similar functions.

### DETAILED DESCRIPTION OF THE ILLUSTRATED EMBODIMENTS

In the following description specific details are set forth describing certain embodiments. It will be apparent, however, to one skilled in the art that the disclosed embodiments may be practiced without some or all of these specific details. The specific embodiments presented are meant to be illustrative, but not limiting. One skilled in the art may realize other material that, although not specifically described herein, is within the scope and spirit of this disclosure.

The surgical laser capsulorhexis system of the present disclosure provides numerous advantages relative to existing technologies, including (1) reduced capital expense; (2) noninvasive; and (3) single-shot in some embodiments described here.

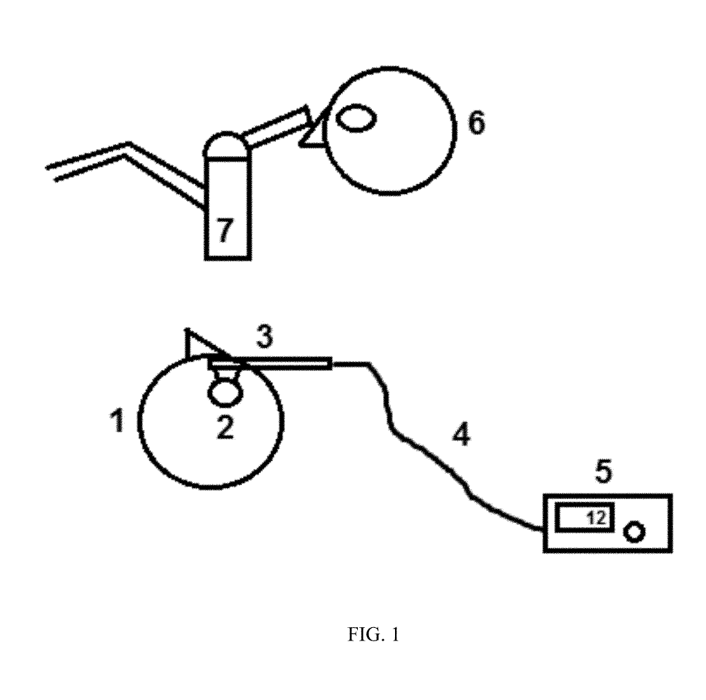

FIG. 1 depicts schematically a capsulorhexis laser and contact lens delivery device. Patient 1 is lying with patient's eye 2 cupped with contact lens 3, which is a laser beam delivery device. Optical fiber 4 connects contact lens assembly to a capsulorhexis laser 5. A surgeon 6 is sitting by the patient conducting surgery and observing it through surgical microscope 7.

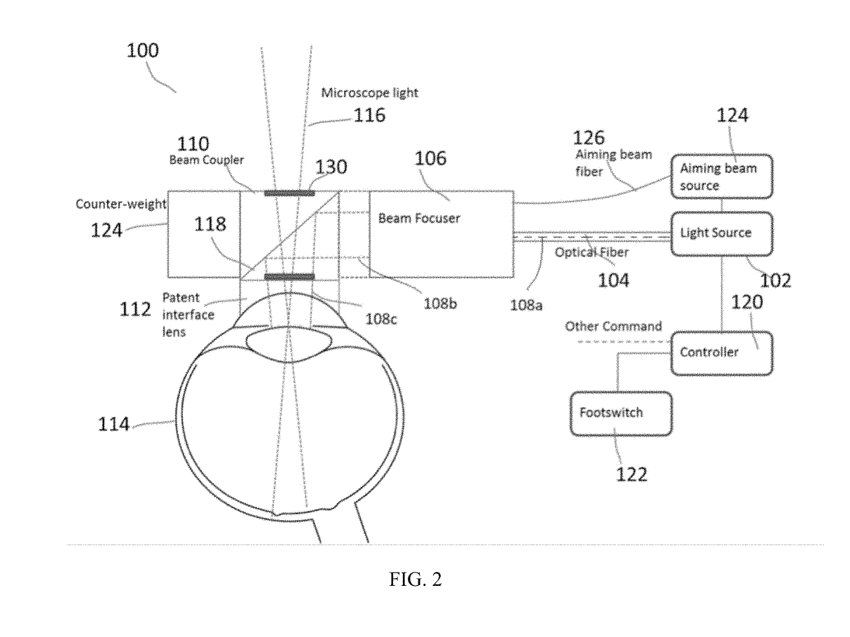

FIG. 2 illustrates a surgical laser capsulorhexis system 100. The surgical laser capsulorhexis system 100 can include a laser source (light source) 102 configured to generate a laser cutting beam 108a. The laser source 102 can have an operating wavelength in the 0.2-2.2 micron range, the 0.7-2.5 micron range, and/or the 1.8-25 micron range. The duration of laser pulse can range from ms (millisecond) to fs (femtosecond). For longer pulses a special dye, absorbing laser radiation, may be used to better localize laser pulse energy release. The surgical laser capsulorhexis system 100 can include a beam guidance system, including an optical fiber 104 and/or free space, configured to guide the laser cutting light beam from the light source. The beam guidance system can also include an aiming beam fiber 126 and/or free space, configured to guide an aiming beam from an aiming beam source 124.

The surgical laser capsulorhexis system 100 can also include a beam focuser 106 configured to guide the laser cutting light beam from the light source 102 and generate a focused laser cutting beam 108b. For laser capsulorhexis the beam focuser 106 can be configured to generate the focused laser cutting beam 108b having a circular pattern. The beam focuser 106 can also be configured to generate the focused laser cutting beam 108b having any other desired one-dimensional or two-dimensional beam patterns, including a line, a spiral, a raster, a cross, a constant-radius asterisk, a multiple-radius asterisk, arcuate shape of various length and angular location, a multiply folded path, and/or other scan patterns. The beam focuser 106 can include one or more of a micro-mirror device, a MEMS (Micro-Electro-Mechanical Systems) based device, a deformable platform, a galvanometer-based scanner, a polygon scanner, a mechanical scanner and/or a resonant PZT (piezoelectric) scanner. The beam focuser 106 can a beam-shaping element. The beam-shaping element can include one or more of a diffractive element, a holographic element, a spatial light modulator, an axicon optical element, a refractive element, bundle fibers, a ring illuminator, a micro-mirror device, a MEMS based device, a deformable platform and/or other beam-shaping element. For example, one or multiple axicon lenses can be used in the beam focuser 106 to generate a ring-shaped two-dimensional beam pattern. Other one-dimensional or two-dimensional beam patterns are also possible by properly selecting and designing the beam-shaping element, such as a holographic element.

The beam focuser 106 can also include focusing optics for defining a depth of focus of the focused laser cutting beam 108b. When present, the focusing optics of the beam focuser 106 can be fixed or adjustable. Adjustable focusing optics or zoom lenses within the beam focuser 106 can facilitate focusing the laser cutting beam 108 onto a region of interest, different focal plan, or with increased resolution and depth-of-field.

The surgical laser capsulorhexis system can also include a beam coupler 110 configured to redirect the focused laser cutting beam 108b towards a patient interface lens 112 configured to guide the redirected focused laser cutting beam 108c into a target region of a procedure eye 114, for example, an anterior lens capsule surface of the procedure eye 114, corneal surface, or other tissue surfaces.

The surgical laser capsulorhexis system 100 and the beam focuser 106 can be configured to provide various cutting ranges on the anterior lens capsule surface of the procedure eye 114, such as between 3 mm and 15 mm, between 4 mm and 8 mm, between 4.5 mm and 7 mm, and/or other desired ranges.

The surgical laser capsulorhexis system can also include a controller 120 configured to take commands from a footswitch 122 or other commands, including one or more from a verbal command from a surgeon or an operator, an electric signal, an optical signal, a magnetic signal from a sensor, a digital command from a processor and/or a combination of the above. The controller 120 is configured to control both the light source 102 and the aiming beam source 124. The footswitch 122 can be connected to the controller 120 with a wire, or through a wireless communication. Push buttons or other control elements may be integrated with handheld laser beam delivery assembly, consisting of focuser, combiner and eye interface.

The aiming beam source 124 emits a visible aiming beam through the aiming beam fiber 126 and beam focuser 106. The visible aiming beam is configured to show a surgeon and/or an operator where the redirected focused laser cutting beam 108c is at the anterior lens capsule surface of the procedure eye 114. The aiming beam source 124 can have an operating wavelength in the 0.4-0.7 micron range.

The surgical laser capsulorhexis system 100 can also include a surgical microscope. The beam coupler 110 can be configured to redirect the focused laser cutting beam 108b into an optical pathway 116 of the surgical microscope light. To redirect the focused laser cutting beam 108b into the target region (for example, the anterior lens capsule surface) of the procedure eye 114 and/or the optical pathway 116 of the surgical microscope, the beam coupler 110 can include a mirror 118. As shown in FIG. 2, the mirror 118 can be tilted such that it is oriented at an oblique angle with respect to each of the focused laser cutting beam 108b and the optical pathway 116 of the surgical microscope. The mirror 118 can include a dichroic mirror, a notch filter, a hot mirror, a beamsplitter, a cold mirror, a beam coupler and/or beam combiner. The mirror 118 can be configured to combine the visible beam of the microscope with the focused laser cutting beam 108b. As a result, the field of view of the focused laser cutting beam 108 and the microscope can overlap partially, overlap completely, (as shown in FIG. 2), or not overlap at all.

The mirror 118 can be configured to reflect the focused laser cutting beam 108b in the wavelength range of the focused laser cutting beam 108b while allowing the visible beam of the microscope to pass through. The mirror 118 can also be configured to reflect at least a portion of the visible aiming beam from the aiming beam source 124, which is coincident with the focused laser cutting beam 108b to facilitate visualization of the focused laser cutting beam 108b, which may be outside of the visible range, such as in the infrared range. For example, the mirror 118 can include a notch filter in the wavelength range of the visible aiming beam such that the visible aiming beam and its reflections from the procedure eye 114 can be partially transmitted through the mirror 118 along the optical pathway 116 to reach the surgical microscope.

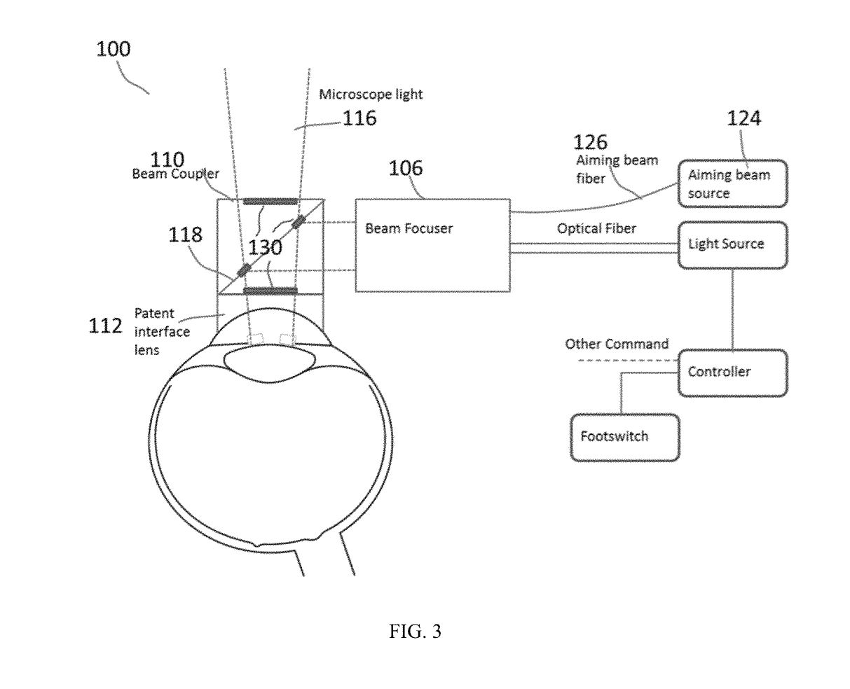

The mirror 118 can also include a high-reflection donut-shaped coating such that both the laser cutting beam and the visible aiming beam are reflected by the mirror 118 and redirected to the anterior lens capsule surface of the procedure eye 114 (FIG. 3).

The beam coupler 110 can be operated with or without a defined optical/opto-mechanical relationship to the surgical microscope. For example, the beam coupler 110 can be maintained separate from and independently positioned relative to the surgical microscope. In such instances, the beam coupler 110 can be a hand-held device, a lens holder, a self-stabilized component or other component.

The beam coupler 110 and the Patient interface lens 112 can be integrated into a common component such that the beam coupler 110 and the Patient interface lens 112 can be collectively, independently positioned relative to the surgical microscope. The surgical laser capsulorhexis system 100 can have the beam coupler 110 and the Patient interface lens 112 integrated into a common component, such as a hand-held device, a lens holder, an adapter, or other component. The Patient interface lens 112 can be separate from, but attachable to the integrated optical block component. The integrated beam coupler 110 and Patient interface lens 112 can be a consumable product configured for use in a single surgical procedure. In another embodiment only patient interface can be single use, while optical delivery system (combiner and focuser) can be re-usable.

The beam focuser 106 and the beam coupler 110 can also be integrated into one optical block component. The surgical laser capsulorhexis system 100 can have the beam focuser 106 and the beam coupler 110 integrated into a common optical block component, such as a hand-held device, a lens holder, an adapter, or other component. The Patient interface lens 112 can be separate from, but attachable to the integrated optical block component. A counter weight 124 can be attached to the beam coupler 110 for better balance and self-stabilization. The integrated beam focuser 106 and beam coupler 110 can be a consumable product configured for use in a single surgical procedure.

The beam focuser 106, the beam coupler 110, the counter weight 124 and the Patient interface lens 112 can all be integrated into a common component. The surgical laser capsulorhexis system 100 can have the beam focuser 106, the beam coupler 110, the counter weight 124 and the Patient interface lens 112 integrated into a common component, such as a hand-held device, a lens holder, an adapter, or other component. The integrated beam focuser 106, beam coupler 110, counter weight 124 and Patient interface lens 112 can be a consumable product configured for use in a single surgical procedure.

Referring to FIG. 3, the beam coupler 110 can be coupled to the surgical microscope, directly or indirectly, such that it has a defined optical/opto-mechanical relationship to the surgical microscope. For example, the beam coupler 110 can be coupled to the surgical microscope by one or more of a suspension system, a mechanical frame, a protruding arm, a conical structure, a magnetic member, an elastic member, and a plastic member. The Patient interface lens 112 can be independently manipulable relative to the procedure eye 114 by a lens-holder—instead of the beam coupler 110—when the beam coupler 110 is coupled to the surgical microscope in a defined optical/opto-mechanical relationship.

The Patient interface lens 112 can be configured to operate spaced from the procedure eye 114, as a non-contact lens, or in contact with the procedure eye 114, as a contact lens. For example, a non-contact Patient interface lens 112 can be configured to operate in a manner similar to a binocular indirect ophthalmoscope (BIOM). The non-contact Patient interface lens 112 can be positioned by one or more of a mechanical coupling to the beam coupler 110, a mechanical coupling to the surgical microscope, a suspension system, and a lens holder. The Patient interface lens 112 can also be a contact lens configured to be contacted to the procedure eye 114. A contact Patient interface lens 112 can be embedded in a stabilizing mechanism, where the stabilizing mechanism can be configured to stabilize the contact Patient interface lens 112 relative to the procedure eye 114. To that end, the stabilizing mechanism can include one or more of a trocar, a counter weight, an air suction, a friction-based system, and an elastic system. Opto-electronic stabilization can be employed via optical elements positions, driven through video and photo displacement sensors.

Other than using an aiming beam source 124 to generate a visible alignment target to indicate the location of the redirected focused laser cutting beam 108, another way is to design some patterns/marks directly on a surface of the beam coupler 110, mirror 118 or patient interface lens 112, located between the procedure eye and the surgical microscope. FIG. 3 illustrates the surgical laser capsulorhexis system 100 having at least one visible alignment target 130 along the visual path of the surgical microscope. By design, the visible alignment target should be located between the procedure eye and the surgical microscope. When the surgeon or operator observes the visible alignment target 130 through the surgical microscope, the covered range of the visible alignment target 130 on the anterior lens capsule surface of the procedure eye 114 is between 3 mm and 15 mm in diameter, between 4 mm and 8 mm in diameter, between 4.5 mm and 7 mm in diameter, and/or other desired ranges.

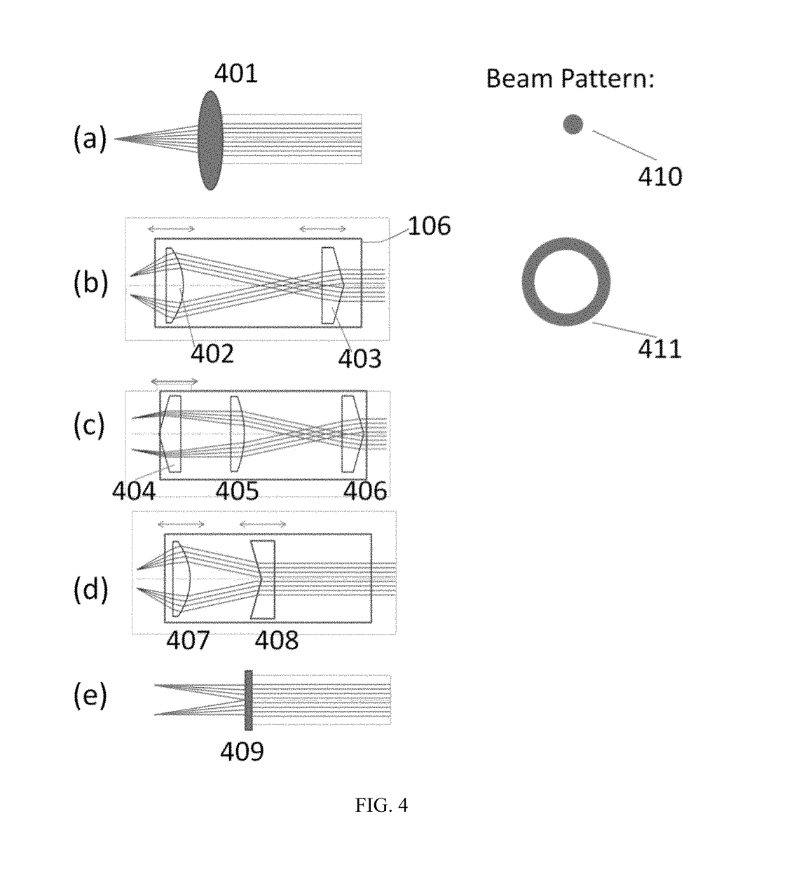

FIG. 4 is a diagram illustrating multiple embodiments of the beam focuser 106 for the surgical laser capsulorhexis system 110. FIG. 4(a) shows a case when a lens 401 is used to focus a laser beam into a single spot beam pattern 410. The single spot beam pattern 410 normally has a dot-like Gaussian beam intensity distribution. This is the case similar to those used in prior arts, such as the femtosecond lasers capsulorhexis system or the nanosecond thermal laser (plus dye) capsulorhexis system mentioned earlier. Those systems have to scan the laser beam sequentially and laterally (across the beam axis) to produce a circular laser cutting on the anterior capsule surface. In another word, a single-shot laser capsulorhexis is not possible. In this patent we refer the dot-like beam pattern as a “one-dimensional” beam pattern, since the system has to scan laterally (in space) and sequentially (in time) to complete the laser capsulorhexis.

FIGS. 4(b)-4(e) show a few embodiments of the beam focuser 106 to generate a ring-shaped or donut-shaped beam pattern 411 at its focal plane by use of a beam-shaping element such as an axicon or a holographic plate. In FIG. 4(b), an axicon 403 takes a beam from right and separates into two parts which are then focused by a lens 402. If both the axicon 403 and lens 402 are moved together (left and right), the focal plane of the beam will be shifted too, while the beam pattern 411 can remain the same. If only one optical component (either axicon 403 or lens 402) is moved, the diameter of the beam pattern can be adjusted accordingly. If the two optical components move away from each other, the beam pattern diameter increases. FIG. 4(c) shows another embodiment where two axicons are used. The beam (from right) is separated by a first axicon 406 and then focused by lens 405. A second axicon 404 is used to adjust the diameter of the beam pattern at the focal plane. If the lens 405 is a zoom lens, liquid crystal lens or a deformable lens, the beam resolution, depth of focus and focal plane can all be adjusted. FIG. 4(d) shows another embodiment where the axicon 408 has an opposite shape (optical power) as axicon 403, 404 and 406. Note that it is possible to combine an axicon and a lens into a single optical component which is normally referred as an “axicon lens.” Either axicon or axicon lens are considered as an axicon optical element.

Here in this patent, the ring-shaped or donut-shaped beam pattern 411 is representative example of a “two-dimensional” beam pattern. Since no sequential or lateral scan of the laser beam is required to complete the laser capsulorhexis. With such a two-dimensional beam pattern, the whole laser capsulorhexis (capsulotomy) can be completed with a single shot of the laser beam.

Furthermore, FIG. 4(e) shows another embodiment where a holographic plate 409 is used to generate a ring-shaped or donut-shaped beam pattern. Holographic plate can be designed to change the diffracted beam through the plate and generate a desired beam pattern at the focal plane. Other two-dimensional beam patterns are also possible, such as a line, an ellipse, a curve, a star, a spiral, a raster, a cross, concentric circles, a constant-radius asterisk, a multiple-radius asterisk, an arcuate shape of various length and angular locations, and a multiply folded path or other more complicated patterns. Other beam-shaping elements can be used for the same purpose too, such as a diffractive element, a spatial light modulator, a refractive element, bundle fibers, a ring illuminator, a micro-mirror device, a MEMS based device, a deformable platform and other beam-shaping element.

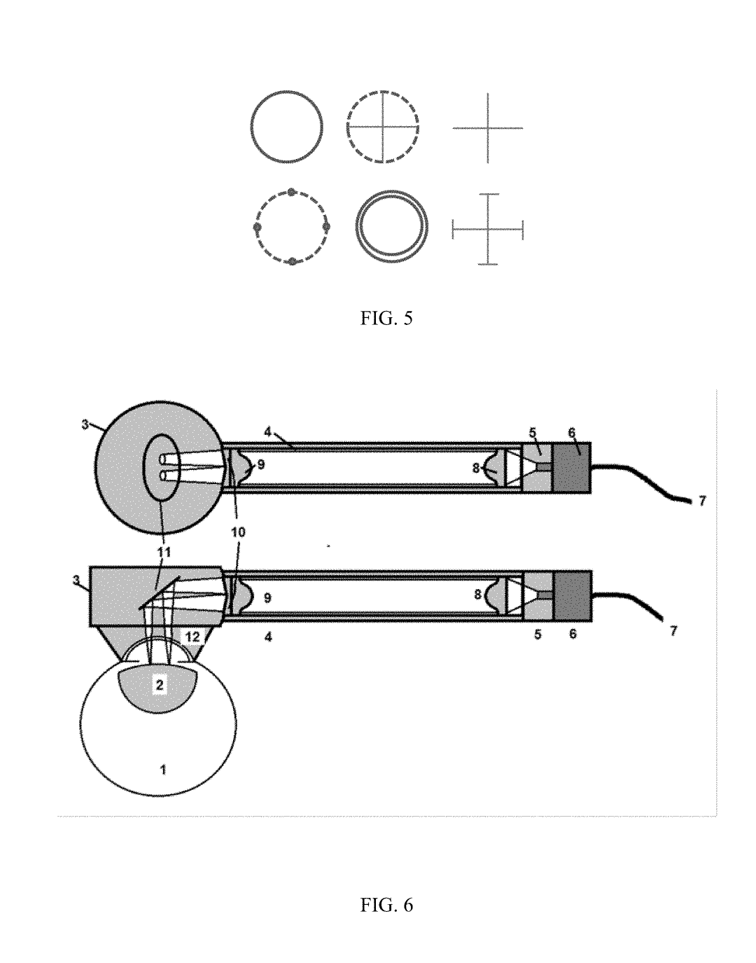

FIG. 5 shows a diagram illustrating a few examples of the visible alignment target 130 for the surgical laser capsulorhexis system 100. Those targets can be designed to have a pattern including a dot, a line, a cross, a circle, a ring, an ellipse, a curve, a star, multiple circles, a spiral, a raster, a dashed circle, a dashed circle with a cross, a dashed circle with dots, a constant-radius asterisk, a multiple-radius asterisk, a multiply folded path, and/or other patterns. As shown in FIG. 3, the visible alignment target 130 can be used to indicate the location of the focused laser cutting beam 108 and allows surgeon to align the surgical laser capsulorhexis system 100 with the target region of the procedure eye 114. In a different embodiment, the mirror 118 can be coated with a donut-shaped coating or mark such that both the laser cutting beam and the visible aiming beam are reflected or partially reflected by the mirror 118 and get redirected to the anterior lens capsule surface of the procedure eye 114. When observed through the surgical microscope by the surgeon, the donut-shaped coating or mark can indicate the location of the focused laser cutting beam 108 and allows surgeon to align the surgical laser capsulorhexis system 100 with the target region of the procedure eye 114. Some other coating patterns are also possible on the mirror 118 to fulfill the same purpose. All patterns shown in FIG. 5 can be generated by coating onto the mirror 118.

FIG. 6 shows a top and side view of a handheld laser delivery system, consisting of an optical fiber 7, connectors 5 and 6, collimating lens 8, focusing lens 9, axicon 10 and beamsplitter 11. Beamsplitter 11 is part of a contact lens 3, applied to eye 1. The ring-shaped focus of laser beam delivery system, including axicon, allows to burn a ring capsulorhexis in one laser shot, provided energy density is sufficient. The laser beam is delivered through the optical fiber 7 which is mounted to the connectors 5 and 6. The beam is then passed through a beam focuser inside a contact lens handle sleeve 4 and a beamsplitter 11. The beam focuser in this embodiment is composed of three optical components: two lenses (collimating lens 8 and focusing lens 9) and one axicon 10. Both the focusing lens 9 and axicon 10 can be moved together left and right to adjust the focus of the focused laser cutting beam onto the anterior surface of human lens 2.

Embodiments as described herein can provide devices, systems, and methods that facilitate real-time, intra-surgical laser capsulorhexis. The examples provided above are exemplary only and are not intended to be limiting. One skilled in the art may readily devise other systems consistent with the disclosed embodiments which are intended to be within the scope of this disclosure. As such, the application is limited only by the following claims.

What is claimed is:

1. A surgical laser capsulorhexis system comprising:
a laser source, configured to generate a laser cutting beam;
a beam guidance system, configured to guide the laser cutting beam from the laser source;
a beam focuser, configured to guide the laser cutting beam from the laser source and to generate a focused laser cutting beam, the focused laser cutting beam having a two-dimensional beam pattern;
a beam coupler, configured to redirect the focused laser cutting beam; and
a patient interface lens, configured to guide the redirected focused laser cutting beam to a tissue surface of a procedure eye.

2. The surgical laser capsulorhexis system of claim 1, wherein the beam focuser includes a beam-shaping element selected from the group consisting an axicon optical element, a diffractive element, a holographic element, a spatial light modulator, a refractive element, bundle fibers, a ring illuminator, a micro-mirror device, a MEMS based device, and a deformable platform.

3. The surgical laser capsulorhexis system of claim 2, wherein the beam focuser is a micro-mirror device, a MEMS based device, a deformable platform, a galvanometer-based scanner, a polygon scanner, or a resonant PZT scanner.

4. The surgical laser capsulorhexis system of claim 1, wherein the two-dimensional beam pattern is selected from the group consisting of a line, a circle, a ring, an ellipse, a curve, a star, a spiral, a raster, a cross, concentric circles, a constant-radius asterisk, a multiple-radius asterisk, an arcuate shape of various length and angular locations, and a multiply folded path.

5. The surgical laser capsulorhexis system of claim 1, wherein the laser source has an operating wavelength in 0.2-2.2 micron, 0.7-2.5 micron, or 18-25 micron wavelength range.

6. The surgical laser capsulorhexis system of claim 1, further comprising a visible alignment target, configured to facilitate alignment of the redirected focused laser cutting beam into a target region of the tissue surface.

7. The surgical laser capsulorhexis system of claim 6, wherein the visible alignment target is generated by an aiming beam source indicating the location of the redirected focused laser cutting beam on a lens capsule surface.

8. The surgical laser capsulorhexis system of claim 6, wherein the visible alignment target covers a range of 3 mm-15 mm in diameter on the tissue surface.

9. The surgical laser capsulorhexis system of claim 6, wherein the visible alignment target is a ring, a line, a cross, a circle, a donut shape, a dot, an arc, a curve, or a star.

10. The surgical laser capsulorhexis system of claim 1, further comprising a surgical microscope,
wherein the beam coupler is configured to redirect the focused cutting beam into an optical pathway of the surgical microscope.

11. The surgical laser capsulorhexis system of claim 10, wherein the visible alignment target is located between the procedure eye and the surgical microscope, on one surface of the beam coupler, or the patient interface lens.

12. The surgical laser capsulorhexis system of claim 1, wherein the beam coupler includes a dichroic mirror, a notch filter, a hot mirror, a beamsplitter, a beam coupler, a beam combiner or a cold mirror in a tilted position.

13. The surgical laser capsulorhexis system of claim 1, wherein the beam coupler is a hand-held device.

14. The surgical laser capsulorhexis system of claim 1, wherein the beam coupler and the patient interface lens are integrated.

15. The surgical laser capsulorhexis system of claim 1, wherein the beam focuser and the beam coupler are integrated into one optical block.

16. The surgical laser capsulorhexis system of claim 10, wherein the beam coupler is coupled to the surgical microscope with a defined optical/opto-mechanical relationship.

17. The surgical laser capsulorhexis system of claim 10, wherein the beam coupler is coupled to the surgical microscope by a suspension system, a mechanical frame, a protruding arm, a conical structure, a magnetic member, an elastic member, or a plastic member.

18. The surgical laser capsulorhexis system of claim 1, wherein the patient interface lens is a non-contact lens, positioned by a mechanical coupling to the beam coupler, a mechanical coupling to the surgical microscope, a suspension system, or a lens holder, or is a contact lens configured to be contacted to the procedure eye.

19. The surgical laser capsulorhexis system of claim 18, wherein the patient interface lens is embedded in a stabilizing mechanism, the stabilizing mechanism configured to stabilize the patient interface lens relative to the procedure eye.

20. The surgical laser capsulorhexis system of claim 19, wherein the stabilizing mechanism includes a trocar, a counter weight, an air suction, a friction-based system, or an elastic system.

21. The surgical laser capsulorhexis system of claim 1, further comprising an footswitch, configured to start laser cutting.

22. The surgical laser capsulorhexis system of claim 1, wherein the aiming beam source has an operating wavelength in 0.4-0.8 micron wavelength range.

23. The surgical laser capsulorhexis system of claim 1, wherein the beam guidance system includes a fiber optical guide and a free space guidance system.

24. A method of performing laser tissue cutting comprising:
generating a laser cutting beam with a two-dimensional beam pattern;
providing a visible alignment target;
aligning the laser cutting beam with a target region of a tissue layer of a procedure eye using the visible alignment target; and
delivering the laser cutting beam to the target region to complete the laser tissue cutting.

25. The method of claim 24, further comprising receiving a corresponding control command to deliver the laser cutting beam.

26. The method of claim 25, further comprising receiving a corresponding control command from a footswitch.

27. The method of claim 24, further comprising:
providing a beam coupler; and
redirecting the laser cutting beam into an optical pathway of a surgical microscope using the beam coupler.

28. The method of claim 24, further comprising generating the visible alignment target by
using an aiming beam source in 0.4-0.8 micron wavelength range, or
generating a visible pattern or mark between the procedure eye and the surgical microscope.

29. A method to perform laser tissue cutting comprising:
generating a laser cutting beam with a two-dimensional beam pattern; and
delivering the laser cutting beam to the target region to complete the laser tissue cutting with a single shot.
